(19) United States
(12) Patent Application Publication
(10) Pub. No.: US 2024/0139490 A1
(43) Pub. Date: May 2, 2024

(54) LUER CONNECTOR FOR VASCULAR ACCESS DEVICES

(71) Applicant: Becton, Dickinson and Company, Franklin Lakes, NJ (US)

(72) Inventor: Raymond P. Feith, Chino Hills, CA (US)

(73) Assignee: Becton, Dickinson and Company, Franklin Lakes, NJ (US)

(21) Appl. No.: 17/977,047

(22) Filed: Oct. 31, 2022

(51) Int. Cl. A61M 39/10

(52) U.S. Cl. CPC A61M 39/1055; A61M 2039/1077; A61M 2039/1088

(57) ABSTRACT

A Luer coupling limits the maximum assembly torque applied to corresponding male and female Luer connector portions of the coupling and another vascular access device (VAD). Corresponding, opposing detents, which are in slidable, mating, abutting engagement contact with each other, limit the maximum applied torque during Luer assembly by slipping past each other when exceeding a desired torque threshold. The torque threshold is selected to be sufficiently high to assure fluid-tight connection, but below a level that induces seizure of the mating Luer connector components. In some embodiments, the opposing detents also create audible and/or tactile vibrations, such as a “clicking” sound as the opposing detents skip over each other. In some embodiments the opposing detents are a pawl and a ratchet.

### TECHNICAL FIELD

The present disclosure generally relates to connectors for vascular access devices (VADs). More particularly, the present disclosure relates to Luer connectors, which limit tightening torque that can be applied to a mating Luer fitting of another VAD while coupling it to the connector.

### BACKGROUND

Vascular access devices (VAD's) are commonly used therapeutic devices and include intravenous (IV) catheters. There are two general classifications of VAD's, peripheral catheters and central venous catheters. Several types of access hubs, ports or valves are coupled to a VAD when delivering a fluid or pharmaceutical. A Luer connector is a common way to couple or join syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure by threading the respective, mating male and female portions of the devices to be coupled.

Standard Luer connectors have a tendency to lock or seize up after mutual coupling of their male and female portions, which makes it difficult to separate them. Seizure is more prevalent when the mating Luer connectors are constructed of the same material, or when the connection was made several days ago. Another potential seizure condition arises when the Luer connector portions are overtightened during their initial coupling by applying excessive rotational torque force to them. Efforts to free seized Luer fittings, such as with pliers or other hand tools, wastes clinician time and can cause patient inconvenience.

### SUMMARY

Luer couplings disclosed herein limit the maximum assembly torque applied to the male and female connector portions. Corresponding, opposing detents, which are in slidable, mating, abutting engagement contact with each other limit the maximum applied torque during Luer assembly by slipping past each other when exceeding a desired torque threshold. The torque threshold is selected to be sufficiently high to assure fluid-tight connection, but below a level that induces seizure of the mating Luer connector components. In some embodiments, the opposing detents also create audible and/or tactile vibrations, such as a “clicking” sound as the opposing detents skip over each other. In this way, the health care practitioner assembling the Luer connectors know that the assembly is secure upon hearing the “clicking” sound. Thus, the opposing detents within the presently disclosed couplings prevent the assembly from being overtightened. In some embodiments, the opposing detents are “one way” detents that allow click-tightening rotation of the Luer connection portions (e.g., during clockwise, relative tightening motion) but lock in place during loosening of the connection portions.

One aspect of the present disclosure pertains to a coupling for a Luer connector, which comprises a first fitting, having a first fluid passage and a first detent. A second fitting is rotatively coupled to the first fitting about a rotational axis. The second fitting has a second fluid passage in fluid communication with the first fluid passage, and a second detent oriented in slidable, mating, abutting engagement with the first detent. A Luer connector is coupled to the first or the second fitting, in fluid communication with its respective first or second fluid passage; and a port is coupled to the other of the first or the second fitting, in fluid communication with its respective first or second fluid passage. The port in turn is coupled to other VADs. When a mating Luer connector of another device is tightened to the Luer connector of the coupling, that relative rotation of the coupled fitting portions causes the mating first and second detents to generate audible and/or tactile vibrations as they slide relative to each other, such as when the Luer portions are tightened above a designated torque threshold.

In some embodiments of the present disclosure, one of the first or the second detent comprises a ratchet and the other of the first or the second detent comprises a biased pawl in slidable, mating, abutting engagement with the ratchet, with the pawl defining a pawl translation axis. In some embodiments, the first and second detent profiles of the mating ratchet and pawl allowing clockwise rotation of the fitting coupled to the Luer connector relative to the fitting coupled to the port upon application of a first rotational torque force threshold but prevents corresponding relative counterclockwise rotation between those fittings. In some embodiments, the mating ratchet and pawl are circumferentially oriented about the rotational axis of the fittings, with the pawl translation axis parallel to the rotational axis. In other embodiments, the ratchet and pawl are oriented on a pawl engagement plane that is normal to the rotational axis of the fittings, with the pawl translation axis parallel to the pawl engagement plane.

Another aspect of the present disclosure pertains to coupling for a Luer connector, which includes a first fitting having a Luer connector in fluid communication with a first portion of a mating, plug-in connector, and a first detent. The coupling also includes a second fitting, having a second portion of the mating, plug-in connector that is rotatively coupled to the first portion thereof about a rotational axis, a port in fluid communication with the first and second portions of the plug-in connector, and a second detent oriented in slidable, mating, abutting engagement with the first detent. Relative rotation of the coupled fitting portions causes the mating first and second detents to generate audible and/or tactile vibrations as they slide relative to each other teach other.

### BRIEF DESCRIPTION OF THE DRAWINGS

Exemplary embodiments of the disclosure are further described in the following detailed description in conjunction with the accompanying drawings, in which:

FIG. 1 is a perspective view of the Luer connector end of a Luer coupling of the present disclosure;

FIG. 2 is a perspective view of the port connector end of a Luer coupling of FIG. 1;

FIG. 3 is an exploded view of the Luer coupling of FIG. 1;

FIG. 4 is another exploded view of the Luer coupling of FIG. 1;

FIG. 5 is an axial, cross-sectional view of the Luer connector of FIG. 1;

FIGS. 6 and 6A are cross-sectional views of the Luer Connector of FIG. 5; and

FIG. 7 is a another embodiment of Luer coupling.

To facilitate understanding, identical reference numerals have been used, where possible, to designate identical elements that are common to the figures. The figures are not drawn to scale.

### DETAILED DESCRIPTION

Aspects of the Luer coupling embodiments disclosed herein limit the maximum assembly torque applied to corresponding male and female Luer connector portions of the coupling and another vascular access device (VAD). Corresponding, opposing detents, which are in slidable, mating, abutting engagement contact with each other, limit the maximum applied torque during Luer assembly by slipping past each other when exceeding a desired torque threshold. The torque threshold is selected to be sufficiently high to assure fluid-tight connection, but below a level that induces seizure of the mating Luer connector components. In some embodiments, the opposing detents also create audible and/or tactile vibrations, such as a “clicking” sound as the opposing detents skip over each other. In some embodiments the opposing detents are a pawl and a ratchet, where the pawl translation axis is parallel to the coupling's rotational axis. In other embodiments the pawl translation axis is a plane that is oriented normal or perpendicular to the rotational axis. In some embodiments, the opposing detents are “one way” detents that allow click-tightening rotation of the Luer connection portions (e.g., during clockwise, relative tightening motion) but lock in place during loosening of the connection portions. Thus, the opposing detents within the presently disclosed couplings prevent the assembly from being overtightened.

In this disclosure, where applicable, a convention is followed wherein the distal end of the device is the end closest to a patient, e.g., for delivery of one or more drugs to the patient through the Luer couplings disclosed herein, and the proximal end of the device is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a flush syringe and can interlock and connect to the female end located on the vascular access device (VAD). A Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification, as applicable, to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways. For example, where the Luer couplings disclosed herein have first and second mating fitting portions shown in one configuration (e.g., mating male and female connections), in other embodiments, the location and orientation of the mating fitting portions are reversed.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

In an exemplary implementation of the embodiments of present disclosure, a first fitting of the Luer connector includes a distal end having a needleless connection Luer connector. In one or more embodiments, the needleless connection includes at least one thread and other features in all combinations allowing it to interface with a corresponding thread or plurality of threads of a corresponding connector.

According to further exemplary implementations of the embodiments of the present disclosure, configuration of structural elements making up the needleless connector include a collar protruding from the distal end of the barrel, the collar comprising at least one thread to connect to the corresponding thread or plurality of threads of a corresponding connector.

According to still further exemplary implementations of the embodiments of the present disclosure, the collar or the needleless connector may bend or elastically deform to allow better interference fit compliance with corresponding connectors.

According to still further exemplary implementations of the embodiments of the present disclosure, the needleless connector may comprise female threads that are sized and have a thread pattern that will engage with a standard ISO594-2 type of male fitting and/or male threads that are sized and have a thread pattern that will engage with a standard ISO594-2 type of female fitting. An example of an ISO594-2 type of fitting is a Q-style fitting.

In one or more embodiments, a male or female connector may be selected from the group consisting essentially of: needle-free connectors, catheter Luer connectors, stopcocks, and hemodialysis connectors. In one or more embodiments, the needleless connector is selected from a Q-Syte connector, MaxPlus, MaxPlus Clear, MaxZero, UltraSite, Caresite, InVision-Plus, Safeline, OneLink, V-Link, ClearLink, NeutraClear, Clave, MicroClave, MicroClave Clear, Neutron, NanoClave, Kendall, Nexus, InVision, Vadsite, Bionector, etc. In one or more embodiments, the male connector or coupling may be an intravenous tubing end, or a stopcock coupled to a port of the Luer coupling.

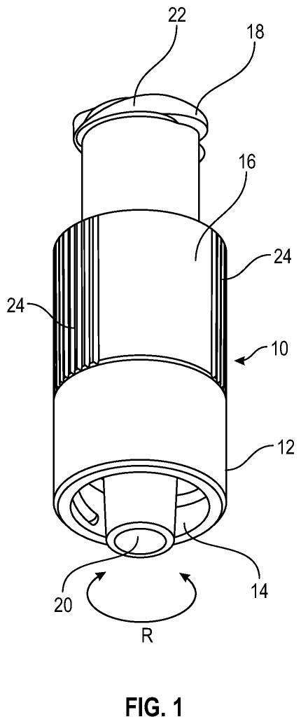

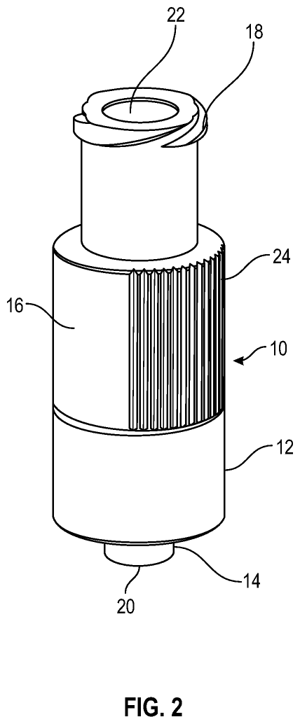

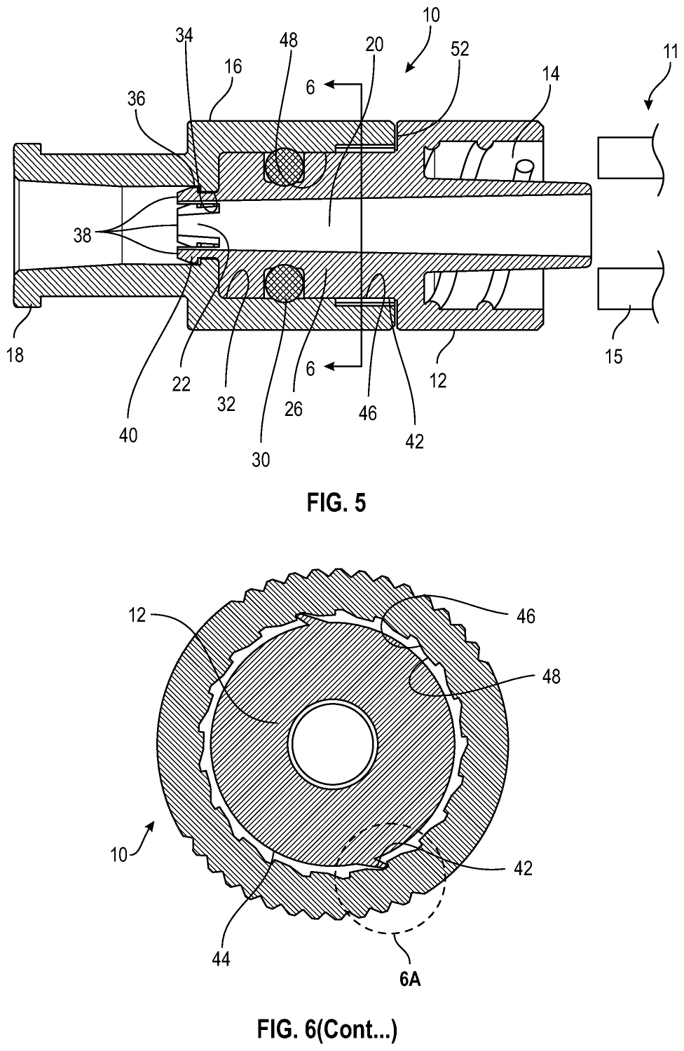

Referring now to the drawings, a first aspect of the present disclosure is shown in FIGS. 1-5. Luer coupling 10 facilitates coupling to a vascular access device (VAD) 11. The Luer coupling 10 has a first fitting 12 whose distal end incorporates a threaded Luer connector 14. In some embodiments the Luer connector 14 is a male connector portion, while in other embodiments it is a female connector portion. As shown in FIG. 5, the Luer connector 14 mates with the corresponding threaded Luer connector 15 of the VAD 11. A second fitting 16 of the Luer connector 10, has a port connector 18 on its proximal end, which as shown facilitates coupling to a catheter. In other embodiments, the port connector 18 is constructed to receive other types of known VAD connection devices, such as Luer connectors, drug infusion ports, and the like. The first 14 and second 16 fittings are rotatively coupled to and in fluid communication with each other, with a first fluid passage 20 of the first fitting in fluid communication with the Luer connector 14 and a second fluid passage 22 of the second fitting. The second fluid passage 22 is in fluid communication with the port 18. An outer circumference of the second fitting 16 defines reeding 24 for grasping by a clinician when attaching the Luer coupling 10 to other VAD's.

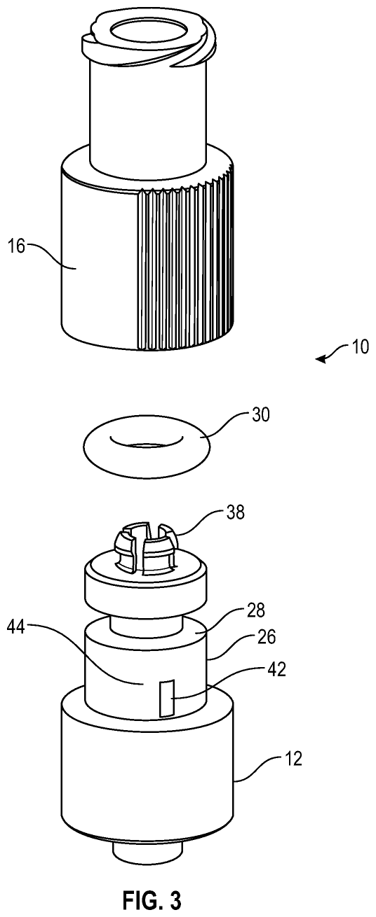

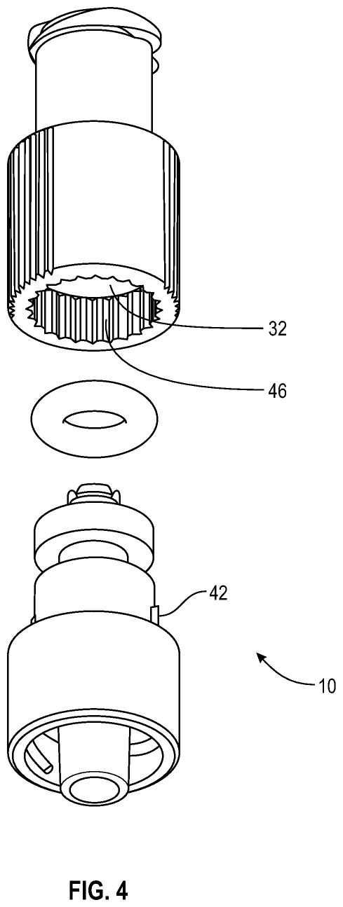

Referring also to the exploded views of FIGS. 3 and 4, the first 12 and second 16 fittings of the Luer coupling 10 are rotatively coupled together by way of a plug-in connector. The Luer coupling 10 incorporates a first plug-in connector 26 in its first fitting 14, which includes an O-ring seat 28 for receipt of O-ring 30. The first plug-in connector 26 mates with a corresponding second plug-in connector 32 in nested orientation about a common, coaxial rotational axis. The O-ring 30 is a circumferential fluid seal interposed between the first 26 and second 32 plug-in connectors, for preventing fluid communication outside of the Luer coupling 10. While the first plug-in connector 26 is a male connector and the second plug-in connector 32 is a mating female connector, in other embodiments they are reversed, so that the female, second plug-in connector is incorporated in the first fitting and the male, first plug-in connector is incorporated in the second fitting.

Referring to FIGS. 3-5, the second plug in connector 32 has a necked bushing 34, which defines an engagement surface 36. A proximal end of the first plug-in connector 26 incorporates a plurality of biased, mating cantilever fingers 38. Each of the cantilever fingers 38 defines a ledge 40 that projects tangentially relative to its cantilever translation axis. Each ledge 40 is in in biased engagement with the engagement surface 36 of the necked bushing 34, thereby preventing relative axial separation of the first 12 and second 16 fittings, while allowing relative rotation therebetween about their common rotational axis.

The first plug-in connector 26 of the first fitting 12 defines a pair of first detents 42 that project radially outwardly from an outer circumferential wall 44 thereof. The second plug-in connector 32 of the second fitting 16 defines a plurality of second detents 46 that project radially inwardly from an inner circumferential wall 48 thereof. The respective first 42 and second 46 detents respectively define outer tips that are aligned with the rotational axis of the Luer coupling 10. Those respective detent tips are oriented in abutting, opposed, slidable engagement with each other, so that relative rotation of the coupled first 12 and second 16 fitting portions causes the mating first 42 and second detents 46 to generate audible and/or tactile vibrations as they slide relative to each other teach other. In the embodiment of FIGS. 1-6, the mating first 42 and second detents 46 generate a “clicking” sound as the slide relative to each other.

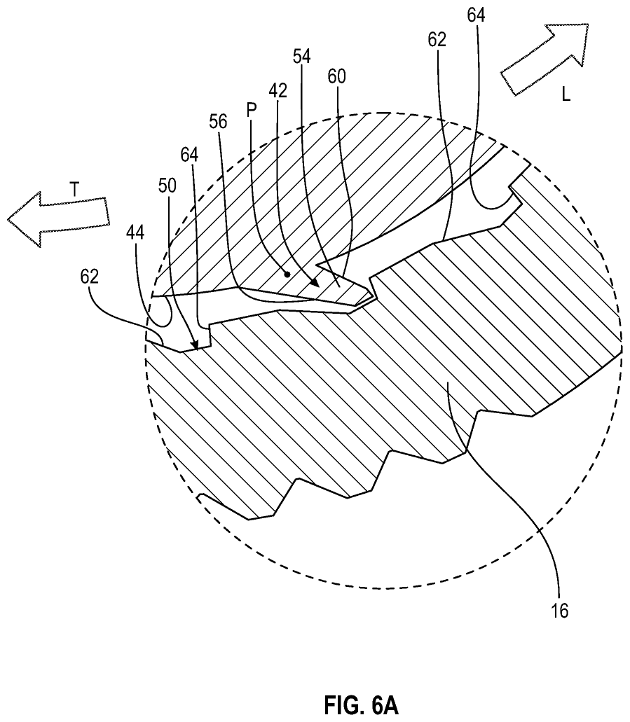

FIG. 6 and enlargement FIG. 6A are cross-sectional views of the Luer coupling 10 taken along 6-6 of FIG. 5, showing the respective first detent 42 and the second detent structure 46. The opposed and abutting first detent 42 and the second detent structure 46 are a pawl and ratchet mechanism, respectively. The inner circumferential wall 48 of the second fitting 16 includes a plurality of circumferentially arrayed ratchet teeth 50 that extend radially inwardly from the inner circumferential wall's axial open end 52. The circumferentially arrayed ratchet teeth 50 engage a pair of pawls 54, oriented at 180 degrees about the outer circumferential wall 44 of the first fitting 16 and projecting radially outwardly therefrom. In other embodiments, the Luer coupling has at least one pawl, while other embodiments have more than two pawls. The profile of each pawl 54 a first pawl slope on a first face 56 and a second pawl slope on a second face 60 that is significantly steeper than the first slope. Each ratchet tooth 50 includes a first slope on a first tooth face 62, and a second slope on a second tooth face 64 that is significantly steeper than the first slope

When the ratchet teeth 50 and the pawls 54 are in in slidable, mating, abutting engagement with each other, the first fitting 12 is rotatable in a first direction relative to the inner second fitting 16 but not rotatable in a second direction opposite the first direction. In particular, the first fitting 12 is rotatable with respect to the second fitting 16 when the first face 56 of each pawl 54 translates about a pawl translation axis P (oriented normal to the drawing figure, which is parallel to the rotational axis of the coupling 10), whereupon it slides over the first slope 62 of each ratchet tooth 50 (see rotational arrow T). Relative rotation of the coupled fitting portions 14 and 14 in the direction T causes the mating first detents (pawls 54) and second detents (ratchet teeth 50) to generate audible and/or tactile vibrations (i.e., a “clicking sound”) as they slide relative to each other. Conversely, contact between the second pawl face 60 of each pawl 50 and the second face 64 of each ratchet tooth 50 prevents rotation of the first fitting 14 relative to the second fitting 16 in an opposite direction (see rotational arrow L). Selectively varying structure and profiles of the ratchet teeth 50 and the mating pawls 54 regulates the applied torque force necessary to cause them to slide relative to each other. The applied torque threshold is selected to assure sufficient fluid sealing of the Luer coupling 10 to any VAD that is screwed into its Luer connector 14, but below an overtightening torque threshold that might cause galling or seizure of the coupling to the VAD.

Referring now to FIGS. 1, 5 and 6, when a clinician screws the auxiliary VAD device 11 having the Luer fitting 15 into its corresponding Luer connector 14 of the Luer coupler 10, it tightens the connection. The clinician optionally may choose to grasp the reeding projections on the outer circumference of the second fitting 16 when screwing in the VAD 11. When tightening torque applied by the clinician in the rotational direction T exceeds a threshold level sufficient to rotate the first fitting 12 relative to the second fitting 16, the pawls 54 and ratchet teeth 50 slide relative to each other, generating the tactile- and audible-detectable “click” sound. The click to tight sound indicates to the treating clinician that the mating Luer connector portions 14 and 15 are in fluid-tight condition and that no further tightening is required. Further effort by the clinician to increase tightening torque on the Luer connector 14 only causes the first fitting 14 to freewheel relative to the second fitting 16 without any further relative tightening of the Luer connector portion 14 and the VAD 11. Thus, it is not possible to overtighten the VAD 11 connection to the Luer coupling 10.

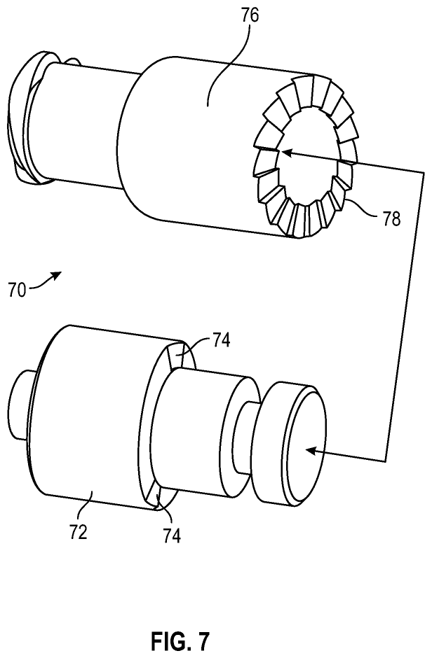

FIG. 7 depicts another embodiment of Luer coupling 70, having a first fitting 72 with first, pawl detents 74 oriented on an axial end thereof and a second fitting 76 with second, ratchet teeth detents 78 on an axial end thereof. The pawl 74 and ratcheting teeth 78 detents are oriented in opposed, slidable, abutting contact with each other on a pawl engagement plane that is normal to the rotational axis of the fittings 72, 76. In the opposing detent orientation of this figure, the pawl 74 translation axis is parallel to said pawl engagement plane. All other structural features of the Luer coupling 72 are similar to those of the Luer coupling 10.

The Luer coupling embodiments disclosed herein are constructed from medical grade materials known to one skilled in the art. In some embodiments, described fitting portions are fabricated with polypropylene polymers. Seals are fabricated with fiber-filled polytetrafluoroethylene (PTFE) polymers and/or with polyisoprene polymers.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are to be interpreted broadly; they encompass direct and indirect mountings, connections, supports, and couplings. Further, “connected” and “coupled” are not restricted to physical, mechanical, or electrical connections or couplings.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A coupling for a Luer connector, comprising:
a first fitting, having a first fluid passage and a first detent; and
a second fitting, rotatively coupled to the first fitting about a rotational axis, the second fitting having a second fluid passage in fluid communication with the first fluid passage, and a second detent oriented in slidable, mating, abutting engagement with the first detent;
a Luer connector coupled to the first fitting or the second fitting, in fluid communication with its respective first or second fluid passage; and
a port coupled to the other of the first fitting or the second fitting, in fluid communication with its respective first or second fluid passage;
so that relative rotation of the coupled fitting portions causes the mating first and second detents to generate audible and/or tactile vibrations as they slide relative to each other.

2. The coupling of claim 1, one of the first or the second detent comprising a ratchet and the other of the first or the second detent comprising a biased pawl in slidable, mating, abutting engagement with the ratchet, the pawl defining a pawl translation axis.

3. The coupling of claim 2, the first and second detent profiles of the mating ratchet and pawl allowing rotation of the fitting coupled to the Luer connector relative to the fitting coupled to the port upon application of a first rotational torque force threshold in a first rotational direction but preventing corresponding relative counter rotation between said fittings in an opposite rotational direction.

4. The coupling of claim 2, the mating ratchet and pawl circumferentially oriented about the rotational axis of the fittings, with the pawl translation axis parallel to said rotational axis.

5. The coupling of claim 2, the ratchet and pawl oriented on a pawl engagement plane that is normal to the rotational axis of the fittings, with the pawl translation axis parallel to said pawl engagement plane.

6. A coupling for a Luer connector, comprising:
a first fitting having a Luer connector in fluid communication with a first portion of a mating, plug-in connector, and a first detent; and
a second fitting, having a second portion of the mating, plug-in connector that is rotatively coupled to the first portion thereof about a rotational axis, a port in fluid communication with the first and second portions of the plug-in connector, and a second detent oriented in slidable, mating, abutting engagement with the first detent;
so that relative rotation of the coupled fitting portions causes the mating first and second detents to generate audible and/or tactile vibrations as they slide relative to each other teach other.

7. The coupling of claim 6, one of the first or second detents comprising a ratchet and the other of the first and second detents comprising a biased pawl in slidable, mating, abutting engagement with the ratchet, the pawl defining a pawl translation axis.

8. The coupling of claim 7, the first and second detent profiles of the mating ratchet and pawl allowing rotation of the fitting coupled to the Luer connector relative to the fitting coupled to the port upon application of a first rotational torque force threshold in a first rotational direction but preventing corresponding relative counter rotation between said fittings in an opposite rotational direction.

9. The coupling of claim 8, the ratchet and pawl circumferentially oriented about the rotational axis of the fittings, with the pawl translation axis parallel to said rotational axis.

10. The coupling of claim 8, the ratchet and pawl oriented on a pawl engagement plane that is normal to the rotational axis of the fittings, with the pawl translation axis parallel to said pawl engagement plane.

11. The coupling of claim 6, the plug-in connector further comprising:
a necked bushing, defining an engagement surface, formed on the first fitting or the second fitting; and
a biased, mating cantilever finger projecting from the other of the first fitting or the second fitting, the finger defining a ledge that projects tangentially relative to its cantilever translation axis, the ledge in biased engagement with the engagement surface of the necked bushing, preventing relative axial separation of the first and second fittings, while allowing relative rotation therebetween about the rotational axis.

12. The coupling of claim 11, further comprising: the first and second portions of the plug-in connector in nested orientation, and a circumferential fluid seal interposed there between, for preventing fluid communication outside of the coupling.

13. The coupling of claim 12, the fluid seal comprising an O-ring.

14. The coupling of claim 11, further comprising:
the first and second portions of the plug-in connector in nested orientation, coaxial with the rotational axis;
the respective first or second detent of the inner one of the nested connector portions projecting radially outwardly from an outer circumferential wall thereof; and
the respective first or second detent of the outer one of the nested connector portions projecting radially inwardly from an inner circumferential wall thereof, in abutting, opposed, slidable engagement with the respective first or second detent of the inner one of the first and second portions.

15. The coupling of claim 14, further comprising:
the outer one of the nested connector portions defining a plurality of symmetric, circumferentially spaced first or second detents, which respectively define outer tips axially aligned with the rotational axis; and
the inner one of the nested connector portions defining at least one first or second detent, which respectively defines an inner tip axially aligned with the rotational axis.

16. The coupling of claim 15, the outer tips comprising a ratchet and the inner tip comprising a biased pawl in slidable, mating, abutting engagement with the ratchet, the pawl defining a pawl translation axis.

17. The coupling of claim 16, one of the first or second detents comprising a ratchet and the other of the first and second detents comprising a biased pawl in slidable, mating, abutting engagement with the ratchet, the pawl defining a pawl translation axis that is parallel to the rotational axis of the fittings.

18. The coupling of claim 17, the first and second detent profiles of the mating ratchet and pawl allowing rotation of the fitting coupled to the Luer connector relative to the fitting coupled to the port upon application of a first rotational torque force threshold in a first rotational direction but preventing corresponding relative counter rotation between said fittings in an opposite rotational direction.

19. The coupling of claim 6, further comprising:
the first and second portions of the plug-in connector in nested orientation, coaxial with the rotational axis;
the first portion of the plug-in connector defining a first axial end oriented along a first axial plane that is normal to the rotational axis of the fitting;
the second portion of the plug-in connector defining a second axial end oriented along a second axial plane that is normal to the rotational axis of the fitting, in opposed orientation with the first axial end;
the respective first or second detent of the first connector portion projecting axially outwardly from the first axial end thereof; and
the respective first or second detent of the second connector portion projecting axially outwardly from the second axial end thereof, in abutting, opposed, slidable engagement with the respective first or second detent of the first connector portion.

20. The coupling of claim 19, one of the first or second detents comprising a ratchet and the other of the first and second detents comprising a biased pawl in slidable, mating, abutting engagement with the ratchet, the pawl defining a pawl translation axis.

21. The coupling of claim 20, the ratchet and pawl oriented on a pawl engagement plane that is normal to the rotational axis of the fittings, with the pawl translation axis parallel to said pawl engagement plane.
